(19) United States
(12) Patent Application Publication
(10) Pub. No.: US 2006/0257475 A1
(43) Pub. Date: Nov. 16, 2006

(54) Stable Sertraline Hydrochloride Formulation and Method

(75) Inventors: Julia ECONOMOU, Dublin, OH (US); Andrea Marie McPhillips, Dublin, OH (US); John Andrew Smoliga, Brookfield, CT (US); Randall Scott Wilson, Dublin, OH (US)

Correspondence Address: MICHAEL P. MORRIS;BOEHRINGER INGELHEIM CORPORATION, 900 RIDGEBURY ROAD, P O BOX 368, RIDGEFIELD, CT, 06877-0368, US

(73) Assignee: Boehringer Ingelheim International GmbH, Ingelheim (DE)

(21) Appl. No.: 11/465,427

(22) Filed: Aug. 17, 2006

(51) Int. Cl. A61K 31/135; A61K 9/20

(52) U.S. Cl. 424/464; 514/657

(57) ABSTRACT

Pharmaceutically stable solid pharmaceutical dosage forms of sertraline hydrochloride Form II and Form V polymorphs formed by direct compression.

### BACKGROUND

1. Field of Invention

The present invention generally relates to stable formulations of sertraline salts manufactured by direct compression process, which stabilize the desired polymorphic form.

2. Discussion of Related Art

Sertraline, (1S-cis)-4-(3,4-Dichlorophenyl)-1,2,3,4-tetrahydro-N-methyl-1-napththalenamine, and in particular its hydrochloride salt (C₁₇H₁₇NCl₂.HCl), having the formula: 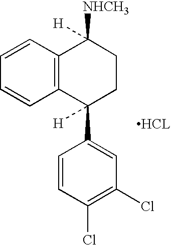
is a useful therapeutic agent, indicated for the treatment and amelioration of numerous physical maladies including, without limitation, depression, anorexia, chemical dependency, obsessive-compulsive disorder, panic disorder, post-traumatic disorder, anxiety-related disorders and premature ejaculation. It is approved for the treatment of one or more of these indications in over sixty countries worldwide.

The daily doses for sertraline, expressed as the free base, range from 25 -200 mg, increasing in 50 mg increments. U.S. Pat. No. 4,536,518 teaches that the compound can be administered in a wide variety of different dosage forms, including, tablets, capsules, lozenges, troches, hard candies, powders, sprays, aqueous suspension, injectable solutions, elixirs, syrups and the like.

Sertraline, and its salts, is hypothesized to cause its pharmacological effects by acting as a selective serotonin reuptake inhibitor (SSRI). Serotonin is known to be an important chemical messenger participating in the transmission of nerve impulses in the brain. Like zimelidine and fluvoxamine, sertraline hydrochloride is believed to selectively block the uptake of serotonin compared to norepinephrine blockade. The drug is a class of compounds that are believed to possess negligible monoamine oxidase inhibition, anticholinergic and psychomotor stimulation activities. Effects upon the cardiovascular system are believed to be minimal. The blockage of pre-synaptosomal uptake of serotonin in the brain is thought to alleviate serotonin abnormalities at adjacent post-synaptic receptor sites. Selective serotonin reuptake inhibitors have been suggested to be useful for treating or preventing numerous conditions including mood disorders, amnetic disorders and age-associated memory impairment, disorders of eating behavior, obesity, cluster headache, migraine, pain, Alzheimer's disease, chronic paroxysmal hemicrania, headache associated with vascular disease, Parkinson's disease, endocrine disorders, vasospasm, hypertension, disorders of the gastrointestinal tract, and chemical dependencies (See, U.S. Pat. 6,727,283).

In solid form sertraline hydrochloride exists in various crystalline forms, so-called polymorphs, each having different physical properties and stability. The conformation and orientation of molecules in the unit cell, which defines a particular polymorphic form of a substance gives rise to such physical properties. Physical properties that are altered by crystalline state include flowability which affects the ease with which a material is handled during processing and rate of dissolution in aqueous fluid which may be rate limiting in respect to the rate at which an orally-administered active ingredient may reach the bloodstream. The polymorphic form may give rise to thermal behavior, measurable by, for example, capillary melting point, thermogravimetric analysis (TGA) and/or differential scanning calorimetry (DSC), which is different from that of amorphous material or another polymorphic form. Distinct solid state ¹³C NMR and infrared spectrographs, and distinct X-ray powder diffraction patterns may be elicited by different polymorphic forms. Polymorphs may be classified as one of two types: enantiotropic (one polymorph can be reversibly changed into another by varying temperature or pressure, e.g., sulfur) or monotropic (one polymorphic form is unstable at all temperatures and pressures, e.g., glyceryl stearates, and non-reversibly changed). Chemical stability, solubility changes and different physical properties due to polymorphism can have an impact on a drug's bioavailability and its development program. Dissolution of a drug particle is controlled by several physicochemical properties including crystal habit, chemical form, particle size, solubility, surface area, and wetting properties. In addition, the physical characteristics of the drug substance itself may lend to preferred methods of manufacture.

U.S. Pat. No. 5,248,699, incorporated herein by reference, describes five crystalline forms of sertraline hydrochloride, designated Form I, Form II, Form III, Form IV and Form V. In this patent the Form I product is reported to have the greatest stability of these five forms, and to be the most suitable for formulation. Additional patents directed to the preparation of Form II of sertraline hydrochloride are U.S. Pat. Nos. 6,452,054, 6,495,721, 6,500,987, 6,872,853, 6,897,340, and 7,067,700,

Additional forms of sertraline hydrochloride are also disclosed in U.S. Pat. Nos. 6,452,054, 6,500,987, 6,858,652 and 7,022,881. U.S. Pat. No. 6,500,987 and WO 00/32551 disclose the existence of new crystal forms of sertraline hydrochloride, denominated Forms VI, VII, VIII, IX and X. Methods for preparation of sertraline hydrochloride polymorphs III, V, VI, VII, VIII, IX and X are also disclosed in U.S. Pat. No. 6,600,073. U.S. Pat. No. 5,734,083 describes a Form T1 as well.

While extensive information is available on the characterization and preparation of several of the polymorphic forms of sertraline, limited information is available on the use of sertraline hydrochloride, or other sertraline salts, in a finished solid pharmaceutical product. Additionally, very limited information is available on formulations able to stabilize sertraline hydrochloride Form II or Form V. A problem commonly seen in formulations of sertraline is the change in polymorphic structure under normal stability conditions. Further, a number of the polymorphic forms have been found to possess pharmaceutically disadvantageous properties. For example, Form I has been reported to be more thermodynamically stable than Forms II, III, IV, and V at room temperature and therefore the most useful form for a commercial pharmaceutical product (See, U.S. Pat. No. 5,248,699). It is has also been reported, however, that Form I is extremely insoluble, hampering adequate bioavailability (See, U.S. Pat. No. 5,734,083).

In formulating any active, such as sertraline, into a tablet or other solid dosage form, one generally attempts to eventuate in a formulation which is storage stable at temperatures and relative humidity levels above those typically encountered. Further, one generally seeks a formulation that does not interfere with the absorption of the active, that is easy to prepare, and which provides for good compressibility.

Solid dosage tablet manufacturing typically takes place using one of three broad methods—direct compression, compression dry granulation, or by particle-bonding wet granulation. Potential benefits of granulation include improvement in powder flow, increase in bulk density, uniform particle size, reduction in punch face adherence and capping tendencies.

Direct compression is used when a group of ingredients can essentially be blended, milled and compressed into tablets. Powders that can be blended and compressed are commonly referred to as directly compressible or as direct-blend formulations. Direct-blending because of its simplicity is quite advantageous. As compared to granulation formulations, direct compression takes fewer steps and therefore less time, as well as less space, materials, and personnel to perform. It also avoids the heat and moisture of wet granulation. Direct compression formulations, as compared to granule formulations, disintegrates more directly into primary particles

Direct compression is frequently not an option because of an inability to uniformly distribute the active through the formulation, the active is poorly compactable in the formulation, the formulation with the active is poorly flowing, exhibits a low density or appropriate filler-binders cannot be found to allow for such a compression. In the case of sertraline hydrochloride Form II and Form V, the low bulk density of the active drug substance and drug loading (approximately 20%) would suggest that the direct compression process would not be a feasible means for manufacture When direct compression is not an option, granulation techniques are options. Granulation, which is either performed dry or wet, is a process of first forming granules from a mix of the active and excipients and then tableting the granules. Granulation formulations disintegrate first into granules and the granules then deaggregate into primary particles.

Dry granulation is used to form granules without using a liquid solution and entails compacting and densifying the powders. Two major modes for compression dry granulation that are conventionally employed are slugging and roller compaction. Slugging consists of dry-blending excipients with the active and then compressing the resultant powder into a large tablet or slug on a compression machine. Roller compaction entails powder compaction wherein compaction pressure and powder feed speed are controlled in a mechanical compression device.

Wet granulation, the process of adding a liquid binder to powders, is one of the most common ways to granulate. Wet granulation allows powder particles to form bonds with one another, the bonds of which may be improved by addition of a binder. Most products that are manufactured as a pharmaceutical are manufactured using the wet granulation process (Tableting & Granulation 2002, Pharmaceutical Technology).

The choice of tableting method is based on many factors including the size of the dose, compactibility or fluidity of the drug, ability to eject, ability to disintegrate, and stability characteristics of the drug. The choice of a tableting method requires a thorough investigation of each ingredient in the formula, the combination of ingredients, and how they work with each other (Tableting & Granulation 2002, Pharmaceutical Technology). Despite the fact that tablets have been made for more than a century, the relationship between ingredients and process conditions with the final properties of tablet are known only anecdotally and are rarely quantified.

Solid dosage formulations typically entail the use of different excipients such as lubricant/glidants, disintegrants, binders and diluents. Lubricants provide lubricity reducing friction between sliding surfaces, and preventing the sticking of the final dosage form, such as a tablet, to surfaces used in the manufacture process. Glidants improve fluidity, that is improve flow, by modifying the interaction between particles. Disintegrants are excipients which oppose the physical forces of particle bonding in the dosage form when placed in an aqueous environment and promote moisture penetration and dispersion of the matrix of the dosage form in dissolution fluids to expose primary drug particles. Binders are excipients which contribute to particle adhesion in a solid formulation, that is they help fuse or link particles to one another. Diluents are used to fill out the intended dosage form weight, i.e. to bulk up the final dosage form.

Due to the many polymorphic forms of sertraline, their convertibility from one polymorphic form to another, and the significant difference between the physical properties of each polymorphic form, the formulation of sertraline into tablets has been found to be quite difficult.

### SUMMARY OF THE INVENTION

The present invention provides formulations of sertraline HCl Form II or Form V, capable of overcoming one or more of the disadvantages of prior art sertraline compositions. In particular, there are provided formulations of sertraline HCl Form II or Form V useful to prepare solid dosage forms, in particular tablets prepared by a direct compression process which have good storage stability and which exhibit numerous formulation advantages. The sertraline HCl Form II and Form V formulations of the present invention are useful for the treatment and amelioration of numerous physical maladies including, without limitation, depression, anorexia, chemical dependency, obsessive-compulsive disorder, panic disorder, post-traumatic disorder, anxiety-related disorders and premature ejaculation.

### DETAILED DESCRIPTION OF THE INVENTION

It has been surprisingly found that Form II and Form V sertraline hydrochloride can be formulated into solid pharmaceutical dosage forms by a direct compression process, and it has also been surprisingly found that the Form II and Form V polymorphs remain in a substantially stable crystalline state in these solid pharmaceutical dosage forms.

In one general embodiment of the present invention, there is provided a solid pharmaceutical dosage form comprising a mixture of sertraline HCl Form II or sertraline HCl Form V and one or more pharmaceutically acceptable carriers or excipients, wherein said solid pharmaceutical dosage form is prepared by a direct compression process wherein the final blend used in the process comprises a mixture of sertraline HCl Form II or sertraline HCl Form V and one or more pharmaceutically acceptable carriers or excipients, said final blend having a bulk density of from about 0.3 to 0.8 g/cc.

By the phrase “bulk density” in the present application is meant the ratio between mass (weight) and bulk volume for the powder blend. Testing for bulk density is typically performed by determining the bulk volume and the weight of a dry powder in a graduated cylinder. The bulk volume in this case takes into consideration the volume of the powder as well as any void spaces that may exist.

By the phrase “direct compression process” in the present application is meant a process for preparing a solid dosage form, such as a tablet, wherein dry, typically powdered, tablet ingredients are blended together in one or more steps to obtain a final blend, and the final blend is then compressed into the solid dosage form. One or more milling steps may be included in the process to aid in the deagglomeration of the blend. This process is to be distinguished from, and does not include, any dry granulation or wet granulation processing steps. Thus, in one example of this direct compression process, the sertraline HCl Form II or Form V is blended together with one or more diluents, disintegrants and glidants to form a pre-blend mixture, this pre-blend mixture is milled and then blended with a lubricant to form the final blend, and this final blend is then compressed into tablets. Multiple variations are possible falling within the scope of a direct compression process, as described in detail herein, and all such variations are covered by the present invention.

By the phrase “final blend” is meant the blend of dry, typically powdered, tablet ingredients that is compressed into the solid dosage form according to the direct compression process of the present invention.

In another embodiment, the sertraline HCl Form II or sertraline HCl Form V in the pharmaceutical dosage form prepared by the direct compression process according to the present invention is substantially stable with respect to its crystalline polymorph form. By the phrase “substantially stable” in the present application is meant that there is no observed change of the sertraline HCl form II or sertraline HCl Form V in the pharmaceutical dosage form into a different polymorphic form when the dosage form is kept under stability conditions of the following temperature and humidity levels: 40° C.±2° C. and 75% RH±5% RH, for 3 months (where “RH” herein stands for relative humidity). Thus, if the sertraline HCl form II or sertraline HCl Form V in the dosage form is substantially stable with respect to its crystalline polymorph form (i.e., no observed change into a different polymorphic form) when the dosage form is kept under temperature and humidity falling within these values, it is covered by the present invention.

The determination of whether there has been an observed change in the polymorphic form can be made using conventional X-Ray powder diffraction (XRPD) techniques, as described in the examples hereinafter, by comparing the XRPD of the initial Form II formulation to the XRPD obtained after storage of the formulation for 3 months under stability conditions.

In another general embodiment of the present invention, there is provided a solid pharmaceutical dosage form comprising a mixture of sertraline HCl Form II or sertraline HCl Form V and one or more pharmaceutically acceptable carriers or excipients, wherein the sertraline HCl Form II or sertraline HCl Form V is substantially stable with respect to its crystalline polymorph form when the dosage form is kept under temperature and humidity conditions of 40° C.±2° C. and 75% RH±5% RH for 3 months, and wherein said solid pharmaceutical dosage form is prepared by a direct compression process wherein the final blend used in the process comprises a mixture of sertraline HCl Form II or sertraline HCl Form V and one or more pharmaceutically acceptable carriers or excipients, said final blend having a bulk density of from about 0.3 to 0.8 g/cc, more preferably from about 0.4 to 0.7 g/cc. This solid pharmaceutical dosage form is preferably a tablet, pellet or granule, most preferably a tablet.

Pharmaceutically acceptable carriers or excipients that might be used in preparing the solid dosage forms of the present invention include, for example, diluents, binders, disintegrants, lubricants and glidants. The selection of particular ingredients is just as important as the selected manufacturing method in order to obtain the proper characteristics of the final blend material and final solid dosage form.

Diluents increase the bulk of a solid pharmaceutical dosage form, and may make a pharmaceutical dosage form containing the composition easier for the patient and care giver to handle. Examples of diluents that might be used in the dosage form include, for example, dibasic calcium phosphate, tribasic calcium phosphate, calcium carbonate, calcium sulfate, microcrystalline cellulose, powdered cellulose, dextrin, dextrose, fructose, lactose, glyceryl palmitosterate, kaolin, magnesium carbonate, magnesium oxide, maltodextrin, mannitol, potassium chloride, sodium chloride, sorbitol, starch, pregelatinized starch, compressible sugar, confectioner's sugar, and hydrogenated vegetable oil, and mixtures thereof.

Solid pharmaceutical compositions that are compacted into a dosage form, such as a tablet, may include excipients whose functions include helping to bind the active ingredient and other excipients together after compression. Suitable binders that might be used in the dosage form include, for example, carbomer (e.g. Carbopol®), ethylcellulose, gelatin, guar gum, cellulose derivatives (such as carobxymethylcellulose, hydroxypropyl methyl cellulose, hydroxyethyl cellulose, hydroxypropyl cellulose, ethyl cellulose, microcrystalline cellulose) and zein, and may include acacia, calcium salts such as calcium phosphate dibasic, calcium sulfate and calcium carbonate), sugars (such as lactose, sucrose, dextrose, glucose, maltodextrin and mannitol), xylitol, polymethacrylates, polyvinylpyrrolidone, starch paste, sorbitol, pregelatinized starch, gum tragacanth, alginic acids (and salts thereof such as sodium alginate), magnesium aluminum silicate, polyethylene glycol, bentonites and the like, and mixtures thereof.

The dissolution rate of a compacted solid pharmaceutical composition in the patient's stomach may be increased by the addition of a disintegrant to the composition. Suitable disintegrants that might be used include, for example, carboxymethylcellulose calcium, carboxymethylcellulose sodium, croscarmellose sodium, crospovidone, magnesium aluminum silicate, methylcellulose, modified cellulose gum (e.g., Ac-Di-Sol®), sodium alginate, and sodium starch glycolate, and mixtures thereof.

Glidants may be added to improve the flowability of a non-compacted solid composition and to improve the accuracy of dosing. Excipients that may function as glidants include colloidal silicon dioxide (e.g. Cab-O-Sil®), magnesium trisilicate, powdered cellulose, starch, talc and tribasic calcium phosphate, and mixtures thereof.

When a dosage form such as a tablet is made by the compaction of a powdered composition, the composition is subjected to pressure from a punch and dye. Some excipients and active ingredients have a tendency to adhere to the surfaces of the punch and dye, which may cause the product to have pitting and other surface irregularities. A lubricant may be added to the composition to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc, zinc stearate, and mixtures thereof.

Additional excipients that may be added include flavoring and coloring agents. The formed tablets may also be film coated using conventional coating materials and techniques. Suitable coating materials include, for example, cellulose acetate phthalate, hydroxypropyl methylcellulose, hydroxypropyl methylcellulose phthalate, polyvinyl alcohol, propylene glycol, titanium dioxide, triacetin and triethyl citrate.

An additional particular embodiment of the present invention includes a solid dosage form comprising about 10 to 25% by weight sertraline HCl Form II or sertraline HCl Form V, about 30 to 80% by weight diluent, about 0.5 to 25% by weight disintegrant, about 0.5 to 3% by weight lubricant and about 0.1 to 5% by weight glidant, and having about 3 to 4 parts diluent to 1 part sertraline HCl Form II or sertraline HCl Form V, wherein the sertraline HCl Form II or sertraline HCl Form V in the dosage form is substantially stable with respect to its crystalline polymorphic form. Preferably, this solid dosage form is a tablet prepared by a direct compression process. All weight percentages set forth in the present application and claims are percent by weight relative to the weight of the final dosage form.

Particular Formulations and Embodiments

A particular preferred formulation of the present invention is a solid pharmaceutical dosage form comprising a mixture of sertraline HCl Form II or sertraline HCl Form V and dibasic calcium phosphate, microcrystalline cellulose, lactose, magnesium stearate, modified cellulose gum and colloidal silicon dioxide, wherein the sertraline HCl Form II or sertraline HCl Form V is substantially stable with respect to its crystalline polymorph form when the dosage form is kept under temperature and humidity conditions of 40° C.±2° C. and 75% RH±5% RH for 3 months. In a preferred embodiment, this solid dosage form is prepared by a direct compression process wherein the final blend used in the process has a bulk density of from about 0.3 to 0.8 g/cc, more preferably from about 0.4 to 0.7 g/cc.

Additional preferred formulations of the present invention include the following formulations (a) to (g), each being substantially stable with respect to its crystalline polymorph form (sertraline HCl Form II or sertraline HCl Form V) when the dosage form is kept under temperature and humidity conditions of 40° C.±2° C. and 75% RH±5% RH for 3 months:
(a) A solid dosage form comprising about 10 to 25% by weight sertraline HCl Form II or sertraline HCl Form V, about 1 to 80% by weight dibasic calcium phosphate, about 1 to 80% by weight microcrystalline cellulose, about 1 to 80% by weight lactose, about 0.5 to 25% by weight modified cellulose gum, 0.5 to 3% by weight magnesium stearate and about 0.1 to 5% by weight colloidal silicon dioxide, and having about 3 to 4 parts diluent (dibasic calcium phosphate+microcrystalline cellulose+lactose) to 1 part sertraline HCl Form II or sertraline HCl Form V.
(b) A solid dosage form comprising about 15 to 20% by weight sertraline HCl Form II or sertraline HCl Form V, about 25 to 45% by weight dibasic calcium phosphate, about 5 to 15% by weight microcrystalline cellulose, about 15 to 25% by weight lactose, about 5 to 15% by weight modified cellulose gum, 0.75 to 2.5% by weight magnesium stearate and about 0.5 to 2% by weight colloidal silicon dioxide, and having about 3 to 4 parts diluent (dibasic calcium phosphate+microcrystalline cellulose+lactose) to 1 part sertraline HCl Form II or sertraline HCl Form V.
(c) A solid dosage form comprising about 19% by weight sertraline HCl Form II or sertraline HCl Form V, about 35% by weight dibasic calcium phosphate, about 13% by weight microcrystalline cellulose, about 20% by weight lactose, about 10% by weight modified cellulose gum, 2% by weight magnesium stearate and about 1% by weight colloidal silicon dioxide.
(d) A solid dosage form comprising 10 mg-100 mg sertraline hydrochloride Form II or sertraline HCl Form V, 1 mg-400 mg dibasic calcium phosphate, 1 mg-100 mg microcrystalline cellulose, 1 mg-300 mg lactose, 0.01 mg-12.00 mg magnesium stearate, 0.1 mg-100 mg modified cellulose gum, and 0.01 mg-15 mg colloidal silicon dioxide and having about 3 to 4 parts diluent (dibasic calcium phosphate+microcrystalline cellulose+lactose) to 1 part sertraline HCl Form II or sertraline HCl Form V.
(e) A solid dosage form comprising 20 mg-30 mg sertraline hydrochloride Form II or sertraline HCl Form V, 25 mg-75 mg dibasic calcium phosphate, 10 mg-30 mg microcrystalline cellulose, 20 mg-40 mg lactose, 2 mg-4 mg magnesium stearate, 5 mg-25 mg modified cellulose gum, and 1.0 mg-3.0 mg colloidal silicon dioxide and having about 3 to 4 parts diluent (dibasic calcium phosphate+microcrystalline cellulose+lactose) to 1 part sertraline HCl Form II or sertraline HCl Form V.
(f) A solid dosage form comprising 40 mg-60 mg sertraline hydrochloride Form II or sertraline HCl Form V, 90 mg-115 mg dibasic calcium phosphate, 30 mg-50 mg microcrystalline cellulose, 50 mg-70 mg lactose, 5 mg-7 mg magnesium stearate, 20 mg-40 mg modified cellulose gum, and 3.0 mg-5.0 mg colloidal silicon dioxide and having about 3 to 4 parts diluent (dibasic calcium phosphate+microcrystalline cellulose+lactose) to 1 part sertraline HCl Form II or sertraline HCl Form V.
(g) A solid dosage form comprising 90 mg-115 mg sertraline hydrochloride Form II or sertraline HCl Form V, 200 mg-220 mg dibasic calcium phosphate, 65 mg-85 mg microcrystalline cellulose, 110 mg-130 mg lactose, 10 mg-14 mg magnesium stearate, 50 mg-70 mg modified cellulose gum, and 6.5 mg-8.5 mg colloidal silicon dioxide and having about 3 to 4 parts diluent (dibasic calcium phosphate+microcrystalline cellulose+lactose) to 1 part sertraline HCl Form II or sertraline HCl Form V.

Preferably, the formulations of the present invention as described hereinabove are those prepared by a direct compression process wherein the final blend used in the process has a bulk density of from about 0.3 to 0.8 g/cc, more preferably from about 0.4 to 0.7 g/cc, and preferably tablets prepared by a direct compression process.

By the term “about” with respect to a recited value in the present application is meant ±10% of the recited value, more preferably ±5%, even more preferably ±1%. For example, “about 20% by weight” means from 18% to 22% by weight, more preferably from 19% to 21%, even more preferably from 19.8% to 20.2%. When the term “about” is used before a range of values, the term “about” is intended to modify both recited end point values. For example, “from about 0.3 to 0.8 g/cc” is equivalent to “from about 0.3 g/cc to about 0.8 g/cc” which generally covers values from 0.27 g/cc to 0.88 g/cc.

Direct Compression Methods

A blended composition may be compressed directly into a compacted dosage form using direct compression techniques. Direct compression produces a more uniform tablet without granules. Carriers and excipients that are well suited for direct compression include those listed hereinabove.

In another embodiment of the present invention, therefore, there is provided a method for manufacturing a solid pharmaceutical dosage form comprising substantially stable sertraline HCl form II or sertraline HCl Form V, said method comprising the steps of: (a) mixing the sertraline HCl Form II or sertraline HCl Form V polymorph with one or more pharmaceutically acceptable carriers or excipients in concentrations sufficient to make the sertraline HCl Form II or sertraline HCl Form V polymorph substantially stable with respect to its crystalline polymorph form in the final solid dosage form to form a blend having a bulk density of from about 0.3 to 0.8 g/cc; and (b) forming a solid dosage form from the blend obtained in step (a) by compression of the blend. The sertraline HCl Form II or sertraline HCl Form V can be obtained according to any of the procedures known in the literature for obtaining this particular polymorphic form of sertraline. See, e.g., the various patent references cited in the Background section herein. The selection of the particular carriers and excipients needed and the relative amounts to be used to obtain a substantially stable dosage form of sertraline hydrochloride Form II or sertraline HCl Form V may be determined by a formulation scientist following the guidance and procedures as set forth herein.

In yet another embodiment of the invention, there is provided a method of manufacture of a direct blend formulation of sertraline hydrochloride Form II or sertraline HCl Form V. Sertraline hydrochloride Form II or sertraline HCl Form V, disintegrant, diluent and optionally a glidant and/or lubricant are preblended such as by in a bin tumbler. The pre-blend is then milled and blended, as for example by passing the pre-blend through a mill equipped with a screen and impeller. The milled and blended material is then admixed with a glidant and/or lubricant to form a blend having a bulk density of from about 0.3 to 0.8 g/cc.

In yet another embodiment of the invention there is disclosed a method for manufacturing a solid pharmaceutical dosage form comprising a sertraline hydrochloride Form II or sertraline HCl Form V, said method comprising the steps of: (a) mixing sertraline hydrochloride Form II or sertraline HCl Form V with a dry blend formulation comprising a disintegrant, a diluent and optionally a glidant and/or lubricant, the blend having a bulk density of from about 0.3 to 0.8 g/cc; and (b) forming a solid pharmaceutical dosage form from the composition of step (a) by compression. Another embodiment is a method comprising: (a) pre-blending a quantum of sertraline hydrochloride Form II or sertraline HCl Form V, a disintegrant, a diluent and optionally a glidant and/or lubricant; (b) milling the pre-blend of step (a) ; (c) admixing to the milled pre-blend of step (b) a quantum of glidant and/or lubricant to form a blend having a bulk density of from about 0.3 to 0.8 g/cc; (d) compressing the blend of step (c) into a solid pharmaceutical dosage form.

In yet another embodiment of the invention, there is provided a formulation comprising sertraline hydrochloride form II or sertraline HCl Form V, dibasic calcium phosphate, microcrystalline cellulose, lactose, magnesium sterate, modified cellulose gum (such as Ac-Di-Sol®) and colloidal silicon dioxide (such as Cab-O-Sil®). Such formulation may advantageously be formed into tablets by direct compression process, for example, by process comprising (a) mixing the sertraline HCl Form II or sertraline HCl Form V with dibasic calcium phosphate, microcrystalline cellulose, lactose, magnesium stearate, modified cellulose gum and colloidal silicon dioxide to form a blend having a bulk density of from about 0.3 to 0.8 g/cc; and (b) compressing the blend obtained in step (a) to form the solid dosage form. In a particular embodiment the blend has a bulk density of from about 0.4 to 0.7 g/cc.

Advantageously tablets made from such formulation may contain 10 mg-100 mg sertraline hydrochloride Form II or sertraline HCl Form V, 1 mg-400 mg dibasic calcium phosphate, 1 mg-100 mg microcrystalline cellulose, 1 mg-300 mg lactose, 0.01 mg-12.00 mg magnesium stearate, 0.1 mg-100 mg modified cellulose gum, and 0.01 mg-15 mg colloidal silicon dioxide. Representative compressed tablets are as set forth below:

### EXAMPLE 1

### Exemplary Direct Compression Formulations

| | 25 mg | 50 mg | 100 mg |
|---|---|---|---|
| Ingredient | Tablet | Tablet | Tablet |
| Sertraline HCl Form II or Form V* | 28 mg | 56 mg | 112 mg |
| Dibasic Calcium Phosphate | 53 mg | 105 mg | 210 mg |
| Dihydrate USP | | | |
| Lactose, NF | 30 mg | 61 mg | 121 mg |
| Macrocrystalline Cellulose | 19 mg | 38 mg | 77 mg |
| Modified Cellulose Gum | 15 mg | 30 mg | 60 mg |
| Colloidal Silicon Dioxide | 2 mg | 4 mg | 8 mg |
| Magnesium Stearate, NF | 3 mg | 6 mg | 12 mg |
| Total Mg/Tablet | 150 mg | 300 mg | 600 mg |

*amount dispensed equivalent to 25, 50 and 100 mg Sertraline

Bulk Density of Formulations of Example 1

The bulk densities of the final blends for the formulations of Example 1 prior to direct compression into tablets were determined using a conventional graduated cylinder method and the results are presented in the table below:

| | Sertraline Form II | Sertraline Form V |
|---|---|---|
| Test | Final Blend | Final Blend |
| Bulk Density (g/cc) | 0.60 g/cc | 0.49 g/cc |

Formulations for direct compression may be used to provide for substantial stability of the polymorphic form at storage under temperatures and humidity conditions of 40° C.±2° C. and 75% RH±5% RH. Formulations of Example 1 may be discerned to stablize the crystalline form of sertraline HCl Form II or sertraline HCl Form V, as illustrated in Example 2 set forth below:

### EXAMPLE 2

### Stability of Crystalline Form II and Form V in the Formulations of Example 1

X-ray crystallographic conditions:

| Instrument | Bruker AXS X-Ray Powder Diffractometer Model D8 | |
|---|---|---|
| | Advance using CuKα radiation (1.54 Å) in Bragg- | |
| | Brentano parafocusing mode, graphite monochromator | |
| | and a scintillation detector. | |
| Procedure: | Scan Type: | Theta-theta, locked couple |
| | Scan Mode: | Step |
| | Scan Range: | 2°-35° 2θ |
| | Step Size: | 0.05° 2θ |
| | Time/Step: | 4 sec. |
| | Tube Powder: | 40 KV, 40 mA |
| | Sample Prep: | Lightly ground, zero background |
| | | small area silicon holder |

Form II Stability Testing Results:

| | | |
|---|---|---|
| Time | Sample | 40° C. ± 2° C./75% ± 5% RH |

Form II, 25 mg Strength

| | | |
|---|---|---|
| Initial | Bottles of 50 tablets | — |
| 3 month | | X-Ray Pattern substantially similar |
| | | to initial sample, with no observed |
| | | change in polymorphic form |

Form II, 50 mg Strength

| | | |
|---|---|---|
| Initial | Bottles of 100 tablets | — |
| 3 month | | X-Ray Pattern substantially similar |
| | | to initial sample, with no observed |
| | | change in polymorphic form |

Form II, 100 mg Strength

| | | |
|---|---|---|
| Initial | Bottles of 100 tablets | — |
| 3 month | | X-Ray Pattern substantially similar |
| | | to initial sample, with no observed |
| | | change in polymorphic form |

Form V Stability Testing Results:

| | | |
|---|---|---|
| Time | Sample | 40° C. ± 2° C./75% ± 5% RH |

Form V, 25 mg Strength

| | | |
|---|---|---|
| Initial | Bottles of 50 tablets | — |
| 1 month | | X-Ray Pattern similar to initial |
| | | sample with no observed change |
| | | in polymorphic form |
| 2 month | | X-Ray Pattern similar to initial |
| | | sample with no observed change |
| | | in polymorphic form |
| 3 month | | X-Ray Pattern similar to initial |
| | | sample with no observed change |
| | | in polymorphic form |

Form V, 50 mg Strength

| | | |
|---|---|---|
| Initial | Bottles of 100 tablets | — |
| 1 month | | X-Ray Pattern similar to initial |
| | | sample with no observed change |
| | | in polymorphic form |
| 2 month | | X-Ray Pattern similar to initial |
| | | sample with no observed change |
| | | in polymorphic form |
| 3 month | | X-Ray Pattern similar to initial |
| | | sample with no observed change |
| | | in polymorphic form |
| Initial | Bottles of 500 tablets | — |
| 1 month | | X-Ray Pattern similar to initial |
| | | sample with no observed change |
| | | in polymorphic form |
| 2 month | | X-Ray Pattern similar to initial |
| | | sample with no observed change |
| | | in polymorphic form |
| 3 month | | X-Ray Pattern similar to initial |
| | | sample with no observed change |
| | | in polymorphic form |

Form V, 100 mg Strength

| | | |
|---|---|---|
| Initial | Bottles of 100 tablets | — |
| 1 month | | X-Ray Pattern similar to initial |
| | | sample with no observed change |
| | | in polymorphic form |
| 2 month | | X-Ray Pattern similar to initial |
| | | sample with no observed change |
| | | in polymorphic form |
| 3 month | | X-Ray Pattern similar to initial |
| | | sample with no observed change |
| | | in polymorphic form |
| Initial | Bottles of 500 tablets | — |
| 1 month | | X-Ray Pattern similar to initial |
| | | sample with no observed change |
| | | in polymorphic form |
| 2 month | | X-Ray Pattern similar to initial |
| | | sample with no observed change |
| | | in polymorphic form |
| 3 month | | X-Ray Pattern similar to initial |
| | | sample with no observed change |
| | | in polymorphic form |

The above data suggests that the direct blend process did not adversely affect the Form II or Form V polymorph of sertraline hydrochloride under the processing conditions studied and that the manufacturing process utilized produces a substantially stable product.

Tableting by direct compression may be performed advantageously by a method such as set forth in Example 3 below.

### EXAMPLE 3

### Method for Producing Sertraline Tablets by Direct Compression

Colloidal silicon dioxide, modified cellulose gum, microcrystalline cellulose NF, sertraline hydrochloride, lactose NF, and dibasic calcium phosphate dihydrate USP are added to a 600 liter bin, which is to be tumbled on a bin tumbler and mixed for 120 revolutions at 8 rpm; (b) the preblend of step (a) is then passed through a comil equipped with a 1.4 mm screen and 1601 impeller, and the blend is mixed for 120 revolutions at 8 rpm; (c) magnesium stearate NF is passed through a no. 20 mesh box screen and then is added to the blend of step (b) and mixed for 40 revolutions at 8 rpm. The resultant material may be directly compressed into tablets, for example by means of a tablet press: 25 mg by 9/32″ round concave beveled with bisect, 50 mg by 11/32″ round concave beveled edge with bisect, and 100 mg by 7/16″ round concave beveled edge with bisect.

As would be understood by one of ordinary skill in the art various equipment and equipment orientations may be employed. For example, without limitation, blending may be by use of bins, v-blenders, cubic blenders, cylindrical blenders, sigma mixers, planetary mixers and ribbon mixers, milling by comils or fitzmills, and various tablet presses may be employed including, without limitation, Fette, Beta, Manesty, Korsch, Kilian, Stokes, IMA, Carver and Colton.

Alternative pharmaceutically acceptable carriers or excipients that might be used in preparing the direct compression tablets include, for example, any of the various diluents, binders, disintegrants, lubricants and glidants set forth previously herein.

While the invention has been described with reference to the certain illustrated embodiments, the words that have been used herein are words of description, rather than words of limitation. Changes may be made, within the purview of the appended claims, without departing from the scope and spirit of the invention in its aspects. Although the invention has been described herein with reference to particular structures, acts, and materials, the invention is not to be limited to the particulars disclosed, but rather can be embodied in a wide variety of forms, some of which may be quite different from those of the disclosed embodiments, and extends to all equivalent structures, acts, and, materials, such as are within the scope of the appended claims.

What is claimed is:

1. A solid pharmaceutical dosage form comprising a mixture of sertraline HCl Form II or sertraline HCl Form V and one or more pharmaceutically acceptable carriers or excipients, wherein said solid pharmaceutical dosage form is prepared by a direct compression process wherein the final blend used in the process comprises a mixture of sertraline HCl Form II or sertraline HCl Form V and one or more pharmaceutically acceptable carriers or excipients, said final blend having a bulk density of from about 0.3 and 0.8 g/cc.

2. A solid pharmaceutical dosage form according to claim 1, wherein the sertraline HCl Form II or sertraline HCl Form V is substantially stable with respect to its crystalline polymorph form when the dosage form is kept under temperature and humidity conditions of 40° C.±2° C. and 75% RH±5% RH for 3 months.

3. The solid dosage form of claim 1 in the form of a tablet.

4. The solid dosage form of claim 1 wherein the pharmaceutically acceptable carriers or excipients are selected from the group consisting of: diluents, binders, disintegrants, lubricants and glidants.

5. The solid dosage form of claim 4 wherein the diluent is selected from the group consisting of: dibasic calcium phosphate, tribasic calcium phosphate, calcium carbonate, calcium sulfate, microcrystalline cellulose, powdered cellulose, dextrin, dextrose, fructose, lactose, glyceryl palmitosterate, kaolin, magnesium carbonate, magnesium oxide, maltodextrin, mannitol, potassium chloride, sodium chloride, sorbitol, starch, pregelatinized starch, compressible sugar, confectioner's sugar, and hydrogenated vegetable oil, and mixtures thereof.

6. The solid dosage form of claim 4 wherein the binder is selected from the group consisting of carbomer, ethylcellulose, gelatin, guar gum, carobxymethylcellulose, hydroxypropyl methyl cellulose, hydroxyethyl cellulose, hydroxypropyl cellulose, ethyl cellulose, microcrystalline cellulose, zein, acacia, calcium salts, sugars, xylitol, polymethacrylates, polyvinylpyrrolidone, starch paste, sorbitol, pregelatinized starch, gum tragacanth, alginic acids and salts thereof, magnesium aluminum silicate, polyethylene glycol, bentonites, and mixtures thereof.

7. The solid dosage form of claim 4 wherein the disintegrant is selected from the group consisting of: carboxymethylcellulose calcium, carboxymethylcellulose sodium, croscarmellose sodium, crospovidone, magnesium aluminum silicate, methylcellulose, modified cellulose gum, sodium alginate, and sodium starch glycolate, and mixtures thereof.

8. The solid dosage form of claim 7 wherein the lubricant is selected from the group consisting of at least one of: magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc, zinc stearate, and mixtures thereof.

9. The solid dosage form of claim 4 wherein the glidant is selected from the group consisting of at least one of: colloidal silicon dioxide, magnesium trisilicate, powdered cellulose, starch, talc, tribasic calcium phosphate, and mixtures thereof.

10. The solid dosage form of claim 1, comprising about 10 to 25% by weight sertraline HCl Form II or sertraline HCl Form V, about 30 to 80% by weight diluent, about 0.5 to 25% by weight disintegrant, about 0.5 to 3% by weight lubricant and about 0.1 to 5% by weight glidant, and having about 3 to 4 parts diluent to 1 part sertraline HCl Form II or sertraline HCl Form V.

11. The solid dosage form of claim 1, comprising sertraline HCl Form II or sertraline HCl Form V, dibasic calcium phosphate, microcrystalline cellulose, lactose, magnesium stearate, modified cellulose gum and colloidal silicon dioxide.

12. The solid dosage form of claim 11, comprising about 10 to 25% by weight sertraline HCl Form II or sertraline HCl Form V, about 1 to 80% by weight dibasic calcium phosphate, about 1 to 80% by weight microcrystalline cellulose, about 1 to 80% by weight lactose, about 0.5 to 25% by weight modified cellulose gum, 0.5 to 3% by weight magnesium stearate and about 0.1 to 5% by weight colloidal silicon dioxide, and having about 3 to 4 parts diluent (dibasic calcium phosphate+microcrystalline cellulose+lactose) to 1 part sertraline HCl Form II or sertraline HCl Form V.

13. The solid dosage form of claim 11, comprising about 15 to 20% by weight sertraline HCl Form II or sertraline HCl Form V, about 25 to 45% by weight dibasic calcium phosphate, about 5 to 15% by weight microcrystalline cellulose, about 15 to 25% by weight lactose, about 5 to 15% by weight modified cellulose gum, 0.75 to 2.5% by weight magnesium stearate and about 0.5 to 2% by weight colloidal silicon dioxide, and having about 3 to 4 parts diluent (dibasic calcium phosphate+microcrystalline cellulose+lactose) to 1 part sertraline HCl Form II or sertraline HCl Form V.

14. The solid dosage form of claim 11, comprising about 19% by weight sertraline HCl Form II or sertraline HCl Form V, about 35% by weight dibasic calcium phosphate, about 13% by weight microcrystalline cellulose, about 20% by weight lactose, about 10% by weight modified cellulose gum, 2% by weight magnesium stearate and about 1% by weight colloidal silicon dioxide.

15. The solid dosage form of claim 11, comprising 10 mg-100 mg sertraline hydrochloride Form II or sertraline HCl Form V, 1 mg-400 mg dibasic calcium phosphate, 1 mg-100 mg microcrystalline cellulose, 1 mg-300 mg lactose, 0.01 mg-12.00 mg magnesium stearate, 0.1 mg-100 mg modified cellulose gum, and 0.01 mg-15 mg colloidal silicon dioxide and having about 3 to 4 parts diluent (dibasic calcium phosphate+microcrystalline cellulose+lactose) to 1 part sertraline HCl Form II or sertraline HCl Form V.

16. The solid dosage form of claim 11, comprising 20 mg-30 mg sertraline hydrochloride Form II or sertraline HCl Form V, 25 mg-75 mg dibasic calcium phosphate, 10 mg-30 mg microcrystalline cellulose, 20 mg-40 mg lactose, 2 mg-4 mg magnesium stearate, 5 mg-25 mg modified cellulose gum, and 1.0 mg-3.0 mg colloidal silicon dioxide and having about 3 to 4 parts diluent (dibasic calcium phosphate+microcrystalline cellulose+lactose) to 1 part sertraline HCl Form II or sertraline HCl Form V.

17. The solid dosage form of claim 1 1, comprising 40 mg-60 mg sertraline hydrochloride Form II or sertraline HCl Form V, 90 mg-115 mg dibasic calcium phosphate, 30 mg-50 mg microcrystalline cellulose, 50 mg-70 mg lactose, 5 mg-7 mg magnesium stearate, 20 mg-40 mg modified cellulose gum, and 3.0 mg-5.0 mg colloidal silicon dioxide and having about 3 to 4 parts diluent (dibasic calcium phosphate+microcrystalline cellulose+lactose) to 1 part sertraline HCl Form II or sertraline HCl Form V.

18. The solid dosage form of claim 11, comprising 90 mg-115 mg sertraline hydrochloride Form II or sertraline HCl Form V, 200 mg-220 mg dibasic calcium phosphate, 65 mg-85 mg microcrystalline cellulose, 110 mg-130 mg lactose, 10 mg-14 mg magnesium stearate, 50 mg-70 mg modified cellulose gum, and 6.5 mg-8.5 mg colloidal silicon dioxide and having about 3 to 4 parts diluent (dibasic calcium phosphate+microcrystalline cellulose+lactose) to 1 part sertraline HCl Form II or sertraline HCl Form V.

19. A solid pharmaceutical dosage form comprising a mixture of sertraline HCl Form II or sertraline HCl Form V, dibasic calcium phosphate, microcrystalline cellulose, lactose, magnesium stearate, modified cellulose gum and colloidal silicon dioxide, wherein the sertraline HCl Form II or sertraline HCl Form V is substantially stable with respect to its crystalline polymorph form when the dosage form is kept under temperature and humidity conditions of 40° C.±2° C. and 75% RH±5% RH for 3 months.

20. The solid dosage form of claim 19, comprising about 10 to 25% by weight sertraline HCl Form II or sertraline HCl Form V, about 1 to 80% by weight dibasic calcium phosphate, about 1 to 80% by weight microcrystalline cellulose, about 1 to 80% by weight lactose, about 0.5 to 25% by weight modified cellulose gum, 0.5 to 3% by weight magnesium stearate and about 0.1 to 5% by weight colloidal silicon dioxide, and having about 3 to 4 parts diluent (dibasic calcium phosphate+microcrystalline cellulose+lactose) to 1 part sertraline HCl Form II or sertraline HCl Form V.

21. The solid dosage form of claim 19, comprising about 15 to 20% by weight sertraline HCl Form II or sertraline HCl Form V, about 25 to 45% by weight dibasic calcium phosphate, about 5 to 15% by weight microcrystalline cellulose, about 15 to 25% by weight lactose, about 5 to 15% by weight modified cellulose gum, 0.75 to 2.5% by weight magnesium stearate and about 0.5 to 2% by weight colloidal silicon dioxide, and having about 3 to 4 parts diluent (dibasic calcium phosphate+microcrystalline cellulose+lactose) to 1 part sertraline HCl Form II or sertraline HCl Form V.

22. The solid dosage form of claim 19, comprising about 19% by weight sertraline HCl Form II or sertraline HCl Form V, about 35% by weight dibasic calcium phosphate, about 13% by weight microcrystalline cellulose, about 20% by weight lactose, about 10% by weight modified cellulose gum, 2% by weight magnesium stearate and about 1% by weight colloidal silicon dioxide.

23. The solid dosage form of claim 19, comprising 10 mg-100 mg sertraline hydrochloride Form II or sertraline HCl Form V, 1 mg-400 mg dibasic calcium phosphate, 1 mg-100 mg microcrystalline cellulose, 1 mg-300 mg lactose, 0.01 mg-12.00 mg magnesium stearate, 0.1 mg-100 mg modified cellulose gum, and 0.01 mg-15 mg colloidal silicon dioxide and having about 3 to 4 parts diluent (dibasic calcium phosphate+microcrystalline cellulose+lactose) to 1 part sertraline HCl Form II or sertraline HCl Form V.

24. The solid dosage form of claim 19, comprising 20 mg-30 mg sertraline hydrochloride Form II or sertraline HCl Form V, 25 mg-75 mg dibasic calcium phosphate, 10 mg-30 mg microcrystalline cellulose, 20 mg-40 mg lactose, 2 mg-4 mg magnesium stearate, 5 mg-25 mg modified cellulose gum, and 1.0 mg-3.0 mg colloidal silicon dioxide and having about 3 to 4 parts diluent (dibasic calcium phosphate+microcrystalline cellulose+lactose) to 1 part sertraline HCl Form II or sertraline HCl Form V.

25. The solid dosage form of claim 19, comprising 40 mg-60 mg sertraline hydrochloride Form II or sertraline HCl Form V, 90 mg-115 mg dibasic calcium phosphate, 30 mg-50 mg microcrystalline cellulose, 50 mg-70 mg lactose, 5 mg-7 mg magnesium stearate, 20 mg-40 mg modified cellulose gum, and 3.0 mg-5.0 mg colloidal silicon dioxide and having about 3 to 4 parts diluent (dibasic calcium phosphate+microcrystalline cellulose+lactose) to 1 part sertraline HCl Form II or sertraline HCl Form V.

26. The solid dosage form of claim 19, comprising 90 mg-115 mg sertraline hydrochloride Form II or sertraline HCl Form V, 200 mg-220 mg dibasic calcium phosphate, 65 mg-85 mg microcrystalline cellulose, 110 mg-130 mg lactose, 10 mg-14 mg magnesium stearate, 50 mg-70 mg modified cellulose gum, and 6.5 mg-8.5 mg colloidal silicon dioxide and having about 3 to 4 parts diluent (dibasic calcium phosphate+microcrystalline cellulose+lactose) to 1 part sertraline HCl Form II or sertraline HCl Form V.

27. A solid pharmaceutical dosage form according to claim 19 in the form of a tablet prepared by a direct compression process wherein the final blend used in the process has a bulk density of from about 0.3 and 0.8 g/cc.

28. A process for preparing a solid pharmaceutical dosage form according to claim 19, said process comprising:
(a) mixing the sertraline HCl Form II or sertraline HCl Form V with dibasic calcium phosphate, microcrystalline cellulose, lactose, magnesium stearate, modified cellulose gum and colloidal silicon dioxide to form a final blend having a bulk density of from about 0.3 and 0.8 g/cc; and
(b) compressing the final blend obtained in step (a) to form the solid dosage form.

29. A processs according to claim 28, wherein said final blend has a bulk density of from about 0.4 and 0.7 g/cc.
